(19) United States
(12) Patent Application Publication
(10) Pub. No.: US 2023/0255700 A1
(43) Pub. Date: Aug. 17, 2023

(54) SURGICAL ROBOTIC ARM, FLEXIBLE ARM AND FLEXIBLE JOINT

(71) Applicant: Precision Robotics (Hong Kong) Limited, Shatin, N.T. (HK)

(72) Inventors: Ping Lai Benny Lo, Shatin, N.T. (HK); Yang Hu, Shatin, N.T. (HK); Chui Lan Woo, Shatin, N.T. (HK)

(21) Appl. No.: 18/003,933

(22) PCT Filed: Jun. 29, 2021
(86) PCT No.: PCT/CN2021/102887

§ 371 Date: Dec. 30, 2022

(30) Foreign Application Priority Data
Jun. 30, 2020 (HK) 22020010222.0

(51) Int. Cl. A61B 34/30; A61B 34/00

(52) U.S. Cl. CPC A61B 34/30; A61B 34/71; A61B 2034/306

(57) ABSTRACT

The present disclosure relates to a surgical robotic arm, a flexible arm, and a flexible joint. The flexible joint (1) comprises two support sections (11) and an articulated section (12) connected between the two support sections (11), the articulated section (12) including a plurality of first turns (121) with contact aided parts (122); wherein, the contact aided parts (122) are oppositely arranged on two sides of each first turn (121), and when the flexible joint (1) is in a bent state, the contact aided parts (122) of adjacent first turns (121) are in contact with each other; and wherein at each first turn (121) of the articulated section (12) are provided a plurality of tendon through-holes (5) for a driving tendon (6) to pass through. Compared with the prior art, the flexible joint of the present disclosure has the merit of less misalignment proneness and better reliability.

### CROSS-REFERENCE TO RELATED APPLICATIONS

[0001A] This Application is a National Stage Application under 35 U.S.C. §371 of PCT Application No. PCT/CN2021/102887, filed Jun. 29, 2021, which claims priority to Hong Kong Patent Application No. 22020010222.0, filed Jun. 30, 2020, the entire contents of which are incorporated by reference herein.

### BACKGROUND OF THE INVENTION

### 1. Field of the Invention

The present disclosure relates to the technical field of medical instruments, particularly to a surgical robotic arm, a flexible arm of the surgical robotic arm, and a flexible joint of the flexible arm.

### 2. Description of the Related Art

Single-port access surgery (SPAS), wherein all instruments and cameras enter the human body through a relatively small single-incision, is getting more and more popular due to its advantage of low-invasion or even non-invasion.

In the prior art, surgical robotic arms are often used to assist in the single-port access surgery. Generally, a surgical robot instrument comprises a base, a positioning arm and a surgical robotic arm, wherein the base is relatively fixed in an operating theatre, and the positioning arm is disposed on the base. The surgical robotic arm can be hold at a desired location relative to the patient via the positioning arm. A variety of terminal manipulators, such as scalpels, jaws, etc., may be provided at an end of the surgical robotic arm. The surgical robotic arm enters the patient’s body at a port to implement surgical operations at a surgical site.

As seen, the design of the surgical robotic arm is the key to implement single-port access surgery. In order to facilitate smooth entrance of a terminal manipulator into the human body, the surgical robotic arm needs to have multiple degrees of bending freedom. Those surgical robotic arms in the prior art obtain these degrees of freedom by means of flexible joint(s). There are many feasible structures for such flexible joint(s) to achieve this function. For example, it is possible to design a flexible joint with multiple “turns”, and to provide articulation at each turn. These turns can be small-scale mechanical joints, such as helical joints, articulated joints, hinge joints and rolling joints, etc. However, the mechanical joints usually have extremely high complexity, high requirement for materials at small scales, high manufacturing costs, and poor reliability, and they are also difficult to clean and disinfect.

In the prior art patent document WO 2017/009604A1, some technical solutions of surgical robotic arms not relaying on mechanical joints are disclosed. Nonetheless, such surgical robotic arms still have the deficiency of complicated structure, high costs, and difficulty in controlling the bending direction and degree, for example.

According to the prior art patent document US 2018/0242824A1, the instrument arm in the Intuitive Surgical Davinci SP system is built by using discrete elements with hinged joints. According to the prior art patent document WO 2017/203231A1, the instrument arm in the Precision Robotics Micro-iges system is also built by using discrete elements with hinged joints. However, the manufacturing and assembling process in these systems could be very costly.

### SUMMARY OF THE INVENTION

In order to solve or at least partially solve the above technical problems, the present disclosure provides a surgical robotic arm, a flexible arm of the surgical robotic arm, and a flexible joint of the flexible arm.

According to one aspect of the present disclosure, a flexible joint is provided, comprising two support sections and an articulated section connected between the two support sections, the articulated section including a plurality of first turns with contact aided parts; wherein the contact aided parts are oppositely arranged on two sides of each first turn, and when the flexible joint is in a bent state, the contact aided parts of adjacent first turn are in contact with each other; and wherein at each first turn of the articulated section are provided a plurality of tendon through-holes for a driving tendon to pass through.

Optionally, the plurality of first turns are connected with each other in a helical manner to form a helical structure.

Optionally, the contact aided parts of adjacent first turns are in rolling contact with each other or mesh with each other.

Optionally, each of the contact aided parts is formed as a smooth protrusion toward an adjacent contact aided part, and is in tangential contact with the adjacent contact aided part at the tip of the protrusion.

Optionally, the contact aided part has a circular or elliptical cross-section.

Optionally, the contact aided part is configured as a cylinder or a cone.

Optionally, each of the contact aided parts is formed as a tooth-like structure meshing with an adjacent contact aided part, or has a polygonal cross-section.

Optionally, an axial centerline of the contact aided part coincides with a circumferential centerline of the first turn.

Optionally, a connection line between at least one pair of tendon through-holes among the plurality of tendon through-holes is perpendicular to an axial centerline of the contact aided part.

Optionally, the flexible joint is integrally formed by 3D printing.

Optionally, the tendon through-hole is designed to be open at the periphery of the first turn.

According to another aspect of the present disclosure, a flexible arm is provided, comprising: at least two flexible joints as described above; and a decoupling section, which is disposed between two adjacent flexible joints and connected to the respective support sections of the two adjacent flexible joints.

Optionally, a tendon route is provided on the decoupling section, and extends to a tendon through-hole provided at a support section of the flexible joint for a driving tendon to pass through.

Optionally, the tendon route is helically provided on a surface of the decoupling section.

Optionally, the tendon route includes a helical wire groove provided on an outer surface of the decoupling section.

Optionally, the decoupling section is configured as a cylinder.

Optionally, the corresponding tendon through-holes of two adjacent flexible joints are offset from each other.

Optionally, the tendon through-holes in the support sections of the two adjacent flexible joints are positioned in such a way that the two adjacent flexible joints bend in an S form in one plane.

Optionally, when an outer surface of the decoupling section is deployed in a plane, the tendon route forms an S-shaped curve in the plane.

Optionally, the flexible arm is integrally formed by 3D printing.

According to yet another aspect of the present disclosure, a surgical robotic arm is provided, comprising: a flexible arm as described above; a terminal manipulator to perform surgical operations; and a wrist joint, two ends of which are respectively connected to the flexible arm and the terminal manipulator, wherein a control cable of the terminal manipulator is accessed from the flexible arm, passes through the wrist joint and connects with the terminal manipulator.

Optionally, the wrist joint comprises: a terminal connecting section for connection with the terminal manipulator; a flexible section including a second turn provided in multiple segments, two ends of which are respectively connected to the terminal connecting section and the flexible arm, wherein at each second turn of the flexible section are oppositely provided at least two pairs of tendon through-holes for a driving tendon to pass through; and a central backbone with elasticity passing through the center of the flexible section, two ends of which are respectively connected to the terminal connecting section and the flexible arm.

Optionally, the flexible arm and the wrist joint are integrally formed by 3D printing.

### BRIEF DESCRIPTION OF THE DRAWINGS

In order to better explain the embodiments of the present disclosure, brief introduction will be made to the associated drawings in the following. It is understood that the drawings described below are only intended to illustrate some embodiments of the present disclosure, and the person skilled in the art can obtain many other technical features and connection relationships not mentioned herein according to these drawings.

FIG. 1 is a schematic view of a surgical robot instrument for performing the robotic laparoscopic surgery;

FIG. 2 is a schematic structural view of a portion of a surgical robotic arm near an terminal manipulator;

FIG. 3 is a schematic view of operation principle of a flexible joint of the surgical robotic arm;

FIG. 4 is a schematic structural view of a flexible joint of another surgical robotic arm;

FIG. 5 is a schematic view of a flexible joint involved in the first embodiment of the present disclosure when stretched;

FIG. 6 is a schematic view of the flexible joint shown in FIG. 5 when bent;

FIG. 7 is a schematic cross-sectional view of a first turn of the flexible joint shown in FIG. 5;

FIG. 8 is a schematic view of a flexible joint involved in the second embodiment of the present disclosure when stretched;

FIG. 9 is a schematic view of a flexible joint involved in the third embodiment of the present disclosure when stretched;

FIG. 10 is a schematic plan view of a contact aided part of a flexible joint involved in the fourth embodiment of the present disclosure;

FIG. 11 is a schematic view of a flexible arm according to the present disclosure when stretched;

FIG. 12 is a schematic view of an outer surface of a decoupling section of the flexible arm shown in FIG. 11 when deployed;

FIG. 13 is a schematic view of another flexible arm according to the present disclosure when stretched;

FIG. 14 is a schematic view of a surgical robotic arm according to the present disclosure when stretched;

FIG. 15 is a schematic view of the surgical robotic arm shown in FIG. 14 when bent;

FIG. 16 is a partially enlarged schematic view of the surgical robotic arm shown in FIG. 14 at the wrist joint.

### REFERENCE SIGNS

1: flexible joint; 11: support section; 12: articulated section; 121: first turn; 122: contact aided part; 1223: contact aided unit; 2: flexible arm; 21: decoupling section; 22: tendon route; 3: wrist joint; 31: flexible section; 311: second turn; 32: central backbone; 33: terminal connecting section; 4: terminal manipulator; 5: tendon through-hole; 6: driving tendon; L1: an axial centerline of the contact aided part; L2: a circumferential centerline of the first turn; L3: a connection line between a pair of tendon through-holes.

### DETAILED DESCRIPTION OF THE EMBODIMENTS

The present disclosure will be described in detail below in conjunction with the accompanying drawings. In order to better illustrate improvements of the present disclosure to a surgical robotic arm, basic principles of the surgical robotic arm and implementation principles of a terminal manipulator will be explained respectively.

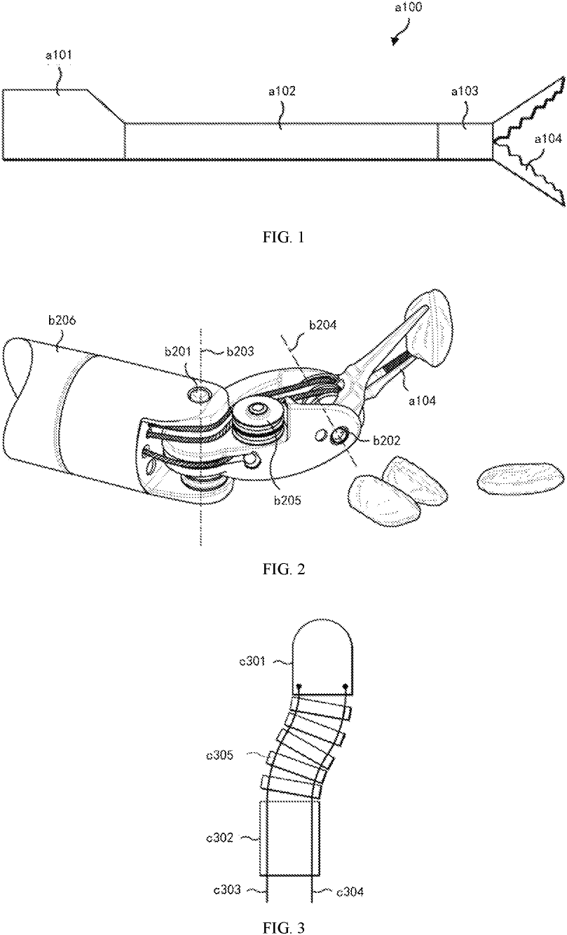

FIG. 1 schematically shows a typical surgical robot instrument a100 for performing the robotic laparoscopic surgery. The surgical robot instrument a100 comprises a base a101, a positioning arm a102, and a surgical robotic arm a103 connected to the base a101 via the positioning arm a102, and a terminal manipulator a104 is provided at an end of the surgical robotic arm a103. In FIG. 1, a pair of zigzag jaws is illustrated as the terminal manipulator a104. The surgical robotic arm a103 allows the terminal manipulator a104 to move with respect to the positioning arm a102, thereby enabling surgical operations within the human body or animal body.

FIG. 2 shows a schematic structural view of a portion of a surgical robotic arm near the terminal manipulator, so as to ease understanding of the specific implementation of the surgical robotic arm. As shown, the terminal manipulator a104 moves relative to the wrist b206 of the surgical robotic arm by means of a pitch joint b201 and a deflection joint b202. The pitch joint b201 enables the terminal manipulator a104 to rotate about a pitch axis b203, and the deflection joint b202 enables the terminal manipulator a104 to rotate about a deflection axis b204. Both the pitch joint b201 and deflection joint b202 are driven by tendons, and a pulley b205 may be used to manage and guide the driving tendons.

FIG. 3 shows a design in which each turn c305 of the flexible joint in the surgical robotic arm is arranged as a concentric ring or disc. In this design, a front end c301 of the flexible joint is used to connect the terminal manipulator a104, and a left driving tendon c303 and a right driving tendon c304 respectively pass through a rear end c302 of the flexible joint and through opposite sides of each turn c305, reaching a front end c301 of the flexible joint. The tension and relaxation of the left driving tendon c303 and the right driving tendon c304 endow the flexible joint with one degree of freedom of movement. When the number of driving tendons is increased to four, the flexible joint will have two degrees of freedom. In this case, however, the terminal manipulator cannot be accurately controlled due to incident misalignment of the turn c305. For example, the left driving tendon c303 and the right driving tendon c304 have the same degree of tension as shown in FIG. 3, but the front end c301 and the rear end c302 of the flexible joint are not collinear.

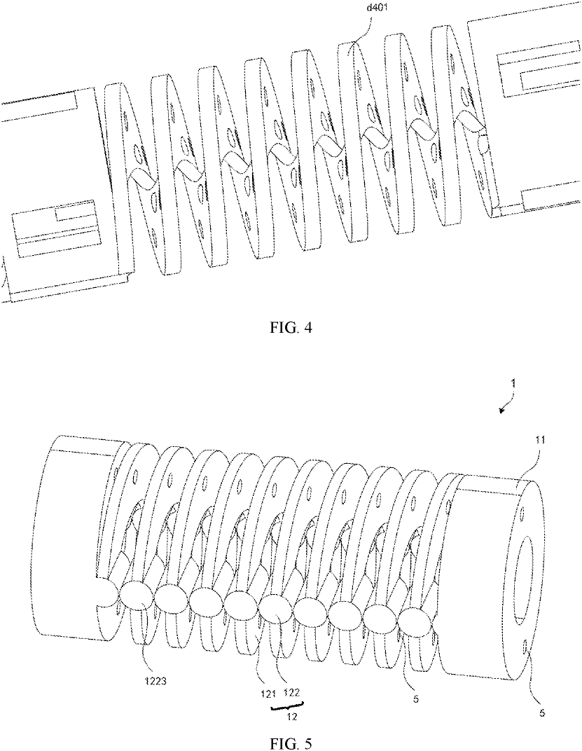

To overcome the deficiency of misalignment, three driving tendons may be used to constrain the flexible joint. However, if each flexible joint requires an additional driving tendon, then the resulting excessive driving tendons will undoubtedly lead to a significant increase in the system complexity and manufacturing costs. In addition, as shown in FIG. 4, it is also possible to form an articulated section composed of the respective turns as a spring d401, and correct the misalignment by the elastic property of the spring d401. However, in the case of a spring structure, if the elastic force of the spring d401 is set to be relatively large, it will be necessary to apply more force on the driving tendons to actuate the joint; and if the elastic force of the spring d401 is set to be relatively small, it is still impossible to effectively correct the misalignment.

In view of the above, the present disclosure provides an improved flexible joint, a flexible arm including the flexible joint, and a surgical robotic arm including the flexible arm, the specific constructions and operation ways of which will be described below with reference to FIGS. 5 to 16.

### I. Flexible Joint

### First Embodiment

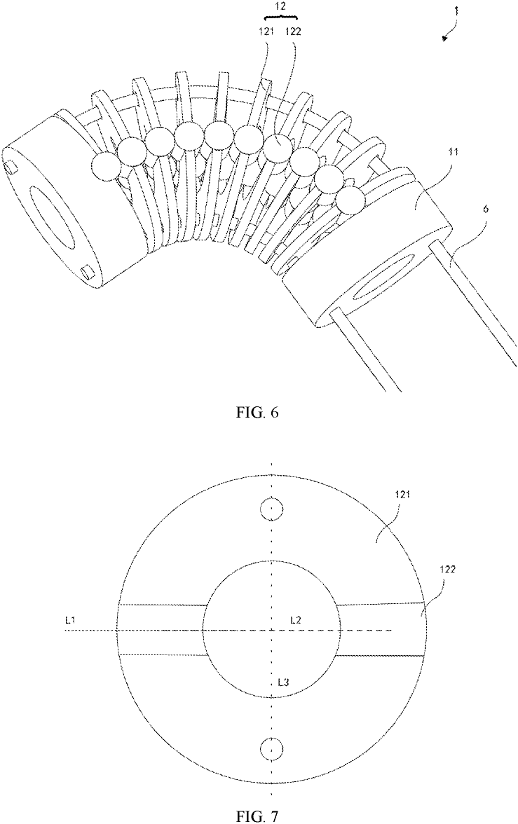

A flexible joint involved in the first embodiment of the present disclosure is illustrated in FIGS. 5 to 7, wherein FIG. 5 is a schematic view of the flexible joint when stretched, FIG. 6 is a schematic view of the flexible joint when bent, and FIG. 7 is a schematic cross-sectional view of a first turn of the flexible joint.

As shown in FIGS. 5 and 6, the flexible joint 1 involved in the first embodiment of the present disclosure comprises two support sections 11 and an articulated section 12 connected between the two support sections 11. The articulated section 12 includes a plurality of first turns 121 with contact aided parts 122. The contact aided parts 122 are oppositely disposed on two sides of each first turn 121. When the flexible joint 1 is in its bent state shown in FIG. 6, the contact aided parts 122 of two adjacent first turns 121 are in contact with each other. A plurality of tendon through-holes 5, through which a driving tendon 6 passes, are oppositely provided at each first turn 121 of the articulated section 12, respectively. The plurality of tendon through-holes 5 may include at least one pair of tendon through-holes 5 provided in pairs along a diameter of the first turn 121. Optionally, one tendon through-hole 5 may be provided at each first turn 121 of the articulated section 12, so that when a driving tendon 6 passing through corresponding tendon through-holes 5 of these first turns 121 is tightened, the flexible joint 1 will bend toward a side where the tendon through-hole 5 is provided.

The supporting section 11 mentioned in the embodiments of the present disclosure refers to a component provided at two ends of the articulated section 12 to play a supporting role. Generally, the support section 11 may have a certain rigidity to enable sufficient support. A tendon through-hole 5 may also be provided at the corresponding positions of the support section 11, so that the driving tendon 6 can pass there-through more smoothly. Additionally, a hollow hole may also be formed in the center of the support section 11 to facilitate passage of various tendons or cables.

Where the support section 11 is rigid, the combination use of rigid support section 11 and flexible articulated section 12 gives it the merit of both groups. That is to say, the rigid support section 11 constrains the movement of the flexible articulated section 12 within the desired 2D space, and has a structural rigidity under a high payload. The flexible articulated section 12 annihilates dominance of non-linear characteristic friction in the rigid support section 11, so that the driving force against the flexible joint 1 is uniformly distributed to each support section 11, and a bending shape with a constant curvature can be formed. This novel design achieves compliance by using a spring-like structure, the torsional and bending forces of which can be more uniformly distributed along a helical beam.

The first turn 121 provided at the articulated section 12 is a key feature to realize the bending movement of the flexible joint 1. As shown in FIG. 6, after a pair of driving tendons 6 pass through the tendon through-holes 5 provided in the support section 11 on one side, each of the first turns 121, and the tendon through-holes 5 provided in the support section 11 on the other side, the driving tendon 6 is relatively fixed to each of the first turns 121, and then bending movement of the articulated section 12 can be driven by the driving tendon 6. Specifically, when the driving tendon 6 on the first side (lower side in FIG. 6) is tightened while the driving tendon 6 on the second side (upper side in FIG. 6) is loosened, the articulated section 12 will bend towards the driving tendon 6 at the first side. With this operation, the flexible joint 1 is given freedom of movement within a plane defined by the pair of driving tendons 6. Compared with the prior art which employs a mechanical joint, the first turn 121 adopted in the present disclosure significantly reduces the complexity of the device.

In an embodiment, the first turn 121 of the flexible joint 1 may be a helical section, that is, a plurality of first turns 121 are connected with each other in a helical manner to form a spring-like helical structure. With this structure, the deficiency of misalignment in the articulated section 12 can be further inhibited. Moreover, since the first turns 121 are connected with each other, the flexible joint 1 will have fewer components and be easier to manufacture and assemble. In addition, the use of a helical articulated section 12 makes it possible to distribute the torsional force and bending force more uniformly on each of the first turns 121, thereby obtaining better flexibility. Optionally, the first turn 121 may also adopt a concentric ring or disc design as shown in FIG. 3, which can still basically achieve the technical objectives of the present disclosure.

In the flexible joint 1 described above, one of essential points lies in the design of the contact aided parts 122. As shown in FIGS. 5 and 6, each contact aided part 122 comprises several contact aided units 1223 disposed oppositely on two sides of each first turn 121. Typically, each contact aided part 122 may be provided with a pair of discrete left and right contact aided units 1223, but more contact aided units 1223 are also feasible. When the flexible joint 1 is in its bent state, the opposite contact aided units 1223 of adjacent first turns 121 are in contact with each other, thereby effectively constraining the bending direction of the articulated section 12.

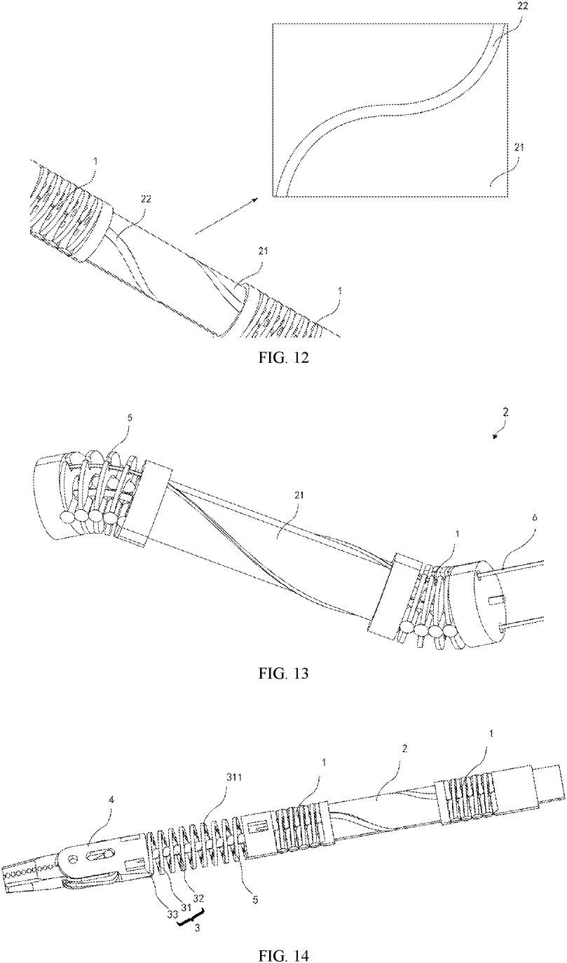

In an embodiment, the tendon through-hole 5 may be designed to be opened at the periphery of the first turn 121. That is, the periphery of the tendon through-hole 5 is not closed, but is open, as shown at the reference numeral 1 in FIG. 13. In this way, an open tendon guiding channel is formed in the flexible joint 1, so that the torque arm of the tendon can be increased when pulling the tendon, and the payload of the instrument arm can be boosted. Therefore, less force is required for bending the flexible joint, offering a larger payload for the instrument arm.

FIG. 7 is a schematic cross-sectional view of the first turn 121 of the flexible joint 1 illustrated in FIG. 5. As shown, the first turn 121 has a circular cross-section; a circular central hole is formed in the center of the first turn 121; and the contact aided parts 122 are diametrically provided on two sides of the hole. An axial centerline L1 of the contact aided parts 122 coincides with a circumferential centerline L2 of the first turn 121, that is, a straight line passing through the cross-section center of the first turn 121. In addition, in the tendon through-hole 5, a connection line L3 between at least one pair of tendon through-holes 5 may be perpendicular to the axial centerline L1 of the contact aided part 122. In this case, a force-bearing surface of the contact aided part 122 will be consistent with the bending direction of the articulated section 12, and therefore better stability can be expected.

It is understood that, the provision of contact aided parts 122 can assist in preventing the misalignment of the joint as shown in FIG. 3. Moreover, the contact aided parts 122 aid contact only, and will not increase the movement resistance of the articulated section 12, so the driving tendon 6 only requires a small force to actuate the flexible joint 1. Thanks to the provision of contact aided units 1223, the first turn 121 can adopt a softer helical section, further lowering requirements for the driving force of the driving tendon 6. With the limit of elastic restoring force of the articulated section 12, movement accuracy of the flexible joint 1 can be improved.

In the flexible joint shown in FIG. 3, the first turn 121 is formed as a concentric ring or disc. In this case, three or more driving tendons 6 are usually needed to ensure the movement accuracy of the flexible joint 1 and prevent misalignment. In the embodiments of the present disclosure, as a contact aided part 122 is formed to constrain the movement of the articulated section 12 within one degree of freedom, a pair of driving tendons 6 would be sufficient to prevent misalignment, thereby reducing the structural complexity of the flexible joint 1.

The flexible joint 1 can be produced by the conventional subtractive manufacturing process or the additive manufacturing process. In the subtractive manufacturing process, some parts are removed from a complete piece of material, such as by creating a specific pattern on a tubular structure and then grooving it to make it bendable. In the additive manufacturing process, flexible joints are built by adding the part layer by layer. It is also possible to use off-the-shelf coil springs to build the flexible joints.

Optionally, the flexible joint 1 of the present disclosure may be integrally formed by 3D printing process. Particularly, when a spring-like helical structure is employed, since the entire flexible joint 1 is connected as a whole, manufacturing the flexible joint 1 by means of an integral molding method will not produce a lot of waste and will eliminate the need for assembling parts one by one, as compared with the traditional manufacturing processes, thereby offering the merit of low costs and high efficiency. Compared with the prior art Intuitive Surgical Davinci SP system and Precision Robotics Micro-iges system, the 3D printing process used in the present disclosure can produce the joint monolithically, and thus can save the manufacturing and assembling cost. For example, as to the flexible joints produced in a subtractive manufacturing process, it is usually using laser cutting to create horizontal slots on an outer surface of a tubular structure and to achieve the compliance based on the bending of a beam-like material, so the slotted tubular structure will have a short design life. In contrast, the flexible joint 1 with helical beams produced by 3D printing has a relatively longer fatigue life. In 3D printing, the same or different materials may be used to form various parts of the flexible joint 1, such as the support section 11, the first turn 121, and the contact aided part 122.

### Second Embodiment

For the contact aided part 122 of the flexible joint 1, the technical objectives of the present disclosure can be achieved as long as the contact aided units 1223 of adjacent first turns 121 can be brought into contact with each other when the articulated section 12 is bent, constraining bending freedom of the articulated section 12. Therefore, the contact aided part 122 may be provided in various feasible shapes and types.

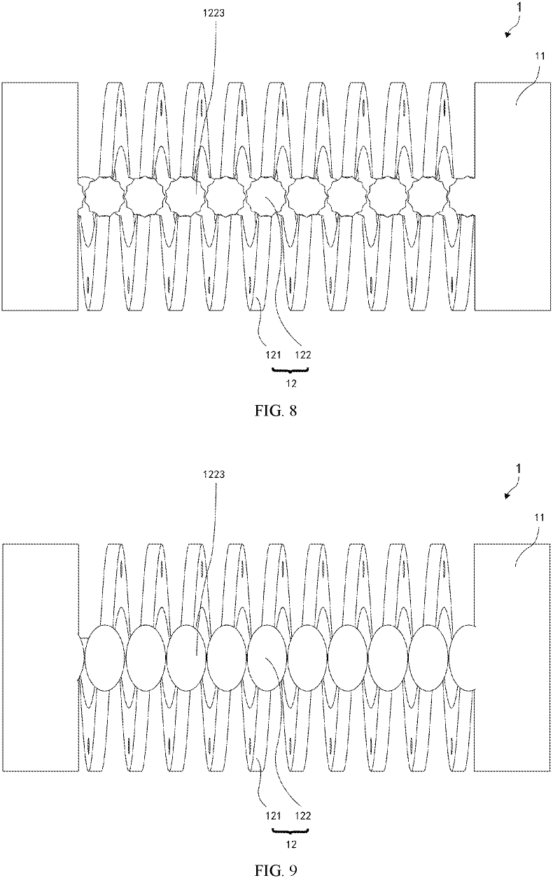

For instance, the contact aided part 122 may be configured as a structure wherein adjacent contact aided units 1223 are capable of meshing with each other, and particularly may be configured as tooth-like structures meshing with one another, as shown in FIG. 8 which schematically illustrates a view of the flexible joint 1 according to the second embodiment when stretched. With a meshing configuration, it is also possible to constrain the freedom of movement of the articulated section 12 while bending it. Among them, the tooth-like structure is able to further prevent deformation of the individual first turns 121. Optionally, it is also possible to design the cross section of the individual contact aided units 1223 as a regular polygon with four or more sides, instead of the tooth-like structure. It is understood that, in addition to the tooth-like structure illustrated in FIG. 8 wherein mutual meshing is obtained along a circumferential direction of the contact aided part 122, a structure wherein mutual meshing is obtained in an axial direction of the contact aided part 122 may be used, which makes it possible to better prevent individual first turns 121 from slipping out of the articulated section 12.

### Third Embodiment

In an embodiment, the contact aided parts 122 of adjacent first turns 121 may be in rolling contact with each other. Compared with the tooth-like structure, the contact aided part 122 with rolling contact has a nearly linear variation in resistance with the bending curve of the articulated section 12, so the bending movement of the articulated section 12 will be smoother, while the requirements for the driving output power of the driving tendon 6 can be reduced.

Where the contact aided part 122 with rolling contact is used, the contact aided part 122 may be embodied in various forms. For example, each contact aided part 122 may be formed as a smooth protrusion toward an adjacent contact aided part 122, and tangentially contact the adjacent contact aided part 122 at the tip of the protrusion. In this case, the contact aided part 122 may have a circular cross-section shown in FIG. 5, or an elliptical cross-section shown in FIG. 9 which is a schematic view of the flexible joint involved in the third embodiment of the present disclosure when stretched.

The elliptical cross-section of the contact aided unit 1223 as shown in FIG. 9 has a smaller size in a direction parallel to the longitudinal axis of the flexible joint 1, and a larger size in a direction perpendicular to the longitudinal axis. In this case, the bending width of the flexible joint 1 is relatively small, and therefore various actions of the terminal manipulator 4 connected to the flexible joint 1 can be adjusted more accurately. Optionally, the contact aided unit 1223 may include an elliptical cross-section with a larger size in the direction parallel to the longitudinal axis of the flexible joint 1 and a smaller size in the direction perpendicular to the longitudinal axis. In this case, the bending width of the flexible joint 1 is relatively large, and therefore it is possible to expand the operation range of the terminal manipulator 4 connected to the flexible joint 1.

### Fourth Embodiment

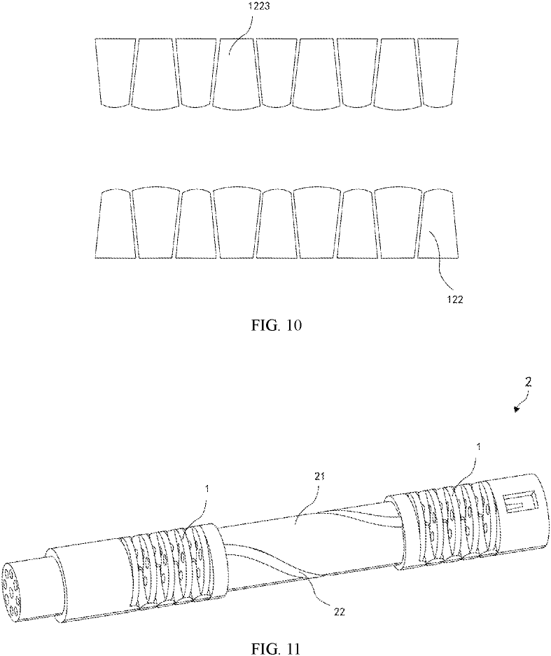

FIG. 10 is a schematic plan view of a contact aided part in a flexible joint involved in the fourth embodiment of the present disclosure. The contact aided part 122 has a circular cross section as shown in FIG. 5, but unlike the cylinder shown in FIG. 5, at least some of the contact aided units 1232 in the contact aided part 122 are configured as cones. That is, the diameter of one contact aided part 122 gradually increases along its axial direction, while the diameter of an adjacent contact aided part 122 contacting therewith gradually decreases in the same direction.

The shapes of cylinders, cones, etc. mentioned in the present disclosure are not strictly defined in a geometric sense. As known in the art, some approximate shapes are often referred to as these shapes. In the present disclosure, a shape can be considered as a “cylinder” or “cone” defined in the claims, as long as it is an approximate shape capable of achieving the technical objectives of the present application. In fact, in order to ensure that overall edges of the flexible joint 1 are smooth and to prevent protruding edges from scratching the inside of the human body, outer surfaces of the contact aided part 122 may be designed to have a certain curvature. Thereby, its end face is no longer a completely flat surface, and therefore it is not strictly a cylinder or a cone. However, these changes in details shall not be regarded as departing from the protection scope of the claims in the present application.

When a cylindrical contact aided unit 1223 is employed, the resistance of the contact aided part 122 to the bending movement of the articulated section 12 remains substantially unchanged as the bending degree of the articulated section 12 increases, and therefore the driving power of the driving tendon 6 can be controlled in an easier manner. In contrast, when a conical contact aided unit 1223 is employed, the shapes of two adjacent contact aided units 1223 will coincide with each other, thereby better preventing the first turns 121 from possibly slipping and misaligning in an axial direction of the cone, leading to an improved working stability of the flexible joint 1.

It is understood that when an elliptical cross section as shown in FIG. 9 is employed, at least some of the contact aided units 1223 in the contact aided part 122 can be formed as an elliptic cylinder or an elliptical cone, as required. Compared with a cylinder, when the contact aided units 1223 of the flexible joint 1 are formed as an elliptical cylinder, the resistance of the contact aided part 122 to the articulated section 12 produced when it bends to various degrees can be accurately adjusted according to the designed curvature, making it better adapt to the output characteristics of the driving power of the driving tendon 6. Also, the difference between an elliptical cone and an elliptical cylinder is similar to that between a cone and a cylinder, and will not be repeated here.

### II. Flexible Arm

FIG. 11 is a schematic view of a flexible arm according to the present disclosure when stretched, and FIG. 12 is a schematic view of an outer surface of a decoupling section of the flexible arm shown in FIG. 11 when deployed. As shown, the flexible arm 2 comprises a flexible joint 1 and a decoupling section 21. The decoupling section 21 is disposed between two adjacent flexible joints 1 and is connected to the respective support sections 11 of the two flexible joints 1. Here, the flexible joint 1 may be the flexible joint described above in the first to fourth embodiments.

When two or more flexible joints 1 are connected in series with each other, it will be necessary to pass a driving tendon 6 for controlling the distal flexible joint 1 through the proximal flexible joint 1, in order to control the distal flexible joint 1. That is, there is a coupling relationship between the proximal flexible joint 1 and the distal flexible joint 1. Accordingly, when the distal flexible joint 1 is controlled by the driving tendon 6, a force applied to the driving tendon 6 will also act on the proximal flexible joint 1 and force it to deform. Especially, when the flexible arm 2 itself forms an S-shaped curve, the direction of deformation will deviate from the bending direction of the proximal flexible joint 1, which may easily cause adverse effects.

For this reason, the flexible arm 2 as shown in FIG. 11 decouples the distal flexible joint 1 and the proximal flexible joint 1 by means of the decoupling section 21, which can weaken or even eliminate the influence of the driving tendon 6 at the distal flexible joint 1 on the proximal flexible joint 1. As such, the flexible arm 2 provided in the embodiments of the present disclosure not only has the advantage of a simple structure, but also can be controlled in an easier manner.

In the embodiment shown in FIG. 11, a tendon route 22 is provided at the decoupling section 21, and extends to a tendon through-hole 5 provided at the support section 11 of a flexible joint 1, so that the driving tendon 6 can penetrate there through. The tendon route 22 can be utilized to guide the arrangement and position of the driving tendons 6, and thus plays an important role in decoupling of the decoupling section 21. Optionally, the tendon route 22 may be configured as a helical wire groove on an outer surface of the decoupling section 21. Also, one or more circumferentially enclosed pipes may be provided at the decoupling section 21 in a helical manner to serve as the tendon route 22. The helical wire grooves and the circumferentially enclosed pipes can be used alone or in combination. In case of a helical wire groove design, the manufacturing cost is lower, and the installation and maintenance of the driving tendon 6 are also simpler and more convenient.

The coupling relationship between the distal flexible joint 1 and the proximal flexible joint 1 is mainly due to the transmission of force between these two flexible joints 1 by the driving tendon 6. By configuring the tendon route 22 in a helical manner on the outer surface of the decoupling section 21, this force can be dispersed to the decoupling section 21 itself, thereby decoupling these two flexible joints 1. It is understood that when the tendon route 22 is provided on the outer surface of the decoupling section 21, the decoupling section 21 may be configured as a cylinder, so as to make the arrangement of the tendon route 22 much easier.

The decoupling section 21 may be a rigid section, the material of which may be consistent with the support section 11. The decoupling section 21 may even be integrally formed with the support section 11. Thereby, the decoupling section 21 can play a good supporting role in the flexible arm 2, so that the flexible arm 2 bends on demand. Further, the entire flexible arm 2 can be integrally formed by 3D printing. When the entire flexible arm 2 is manufactured in an integral manner, there is no need to assemble the flexible joint 1 and decoupling section 21 in sequence. As a result, the complexity of the flexible arm 2 is reduced, the reliability thereof is improved, and the integral flexible arm 2 can possess more excellent mechanical properties and longer service life. In 3D printing, the same or different materials may be used to form various parts of the flexible arm 2, such as the flexible joint 1 and the decoupling section 21.

The corresponding tendon through-holes 5 of two adjacent flexible joints 1 can be offset from each other, wherein the term “corresponding tendon through-holes 5” refers to those tendon through-hole 5 in the two adjacent flexible joints 1 penetrated by the same driving tendon(s) 6. When two tendon through-holes 5 are offset from each other, it will be easier to arrange the driving tendon 6 in a helical shape. In the embodiment shown in FIG. 11, the corresponding tendon through-holes 5 of two adjacent flexible joints 1 are offset from each other by 360 degrees. That is, the driving tendon 6 entering a tendon through-hole 5 of one flexible joint 1 6 winds around the decoupling section 21 for a full circle, and then exist through a tendon through-hole 5 of the other flexible joint 1, so that the two flexible joints 1 are still arranged on the same straight line. As shown in FIG. 12, when the surface of the decoupling section 21 shown in FIG. 11 is deployed in a plane, the tendon route 22 forms an S-shaped curve in the plane. Conventional helical tendon routes 22 are approximately linear in a plane when deployed. When the tendon route 22 forms an S-shaped curve in a plane when deployed, curvatures of the sections will vary along the S-shaped curve, and therefore the resistance of the surface of the tendon route 22 to the driving tendon 6 increases, thereby leading to an improved decoupling ability of the decoupling section 21.

In an embodiment shown in FIG. 13, another flexible arm according to the present disclosure is shown as having a substantially S-shaped profile when stretched. Here, the tendon through-holes 5 in the support sections 11 of two adjacent flexible joints 1 are positioned in such a way that the offset angle of the corresponding tendon through-holes 5 is about 180 degrees. That is, the driving tendon 6 entering a tendon through-hole 5 of one flexible joint 1 winds around the decoupling section 21 for a half circle, and then exist through a tendon through-hole 5 of the other flexible joint 1, thereby bending the two flexible joints 1 in an S shape in one plane. Optionally, the tendon through-holes 5 in the support sections 11 of two adjacent flexible joints 1 may be offset by angles other than 180 degrees and 360 degrees, such as by 90 or 45 degrees. In this case, the distal flexible joint 1 and the proximal flexible joint 1 will not be coplanar.

As for the single-port access surgery, all surgical instruments enter the human body through one single operating channel, and therefore it is difficult to achieve coordinated operations within a narrow working space. Where the flexible arm 2 bends in an S shape in one plane, or the distal flexible joint 1 and the proximal flexible joint 1 are not coplanar, it is possible to better enable various coordinated operations of the terminal manipulator 4 installed at a distal end of the flexible arm 2 within a narrow working space.

### III. Surgery Robotic Arm

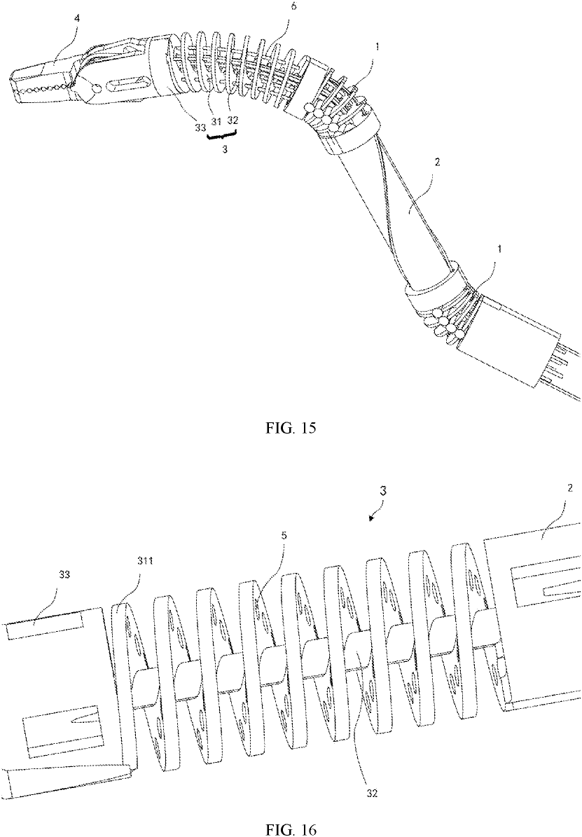

FIG. 14 schematically illustrates a view of a surgical robotic arm according to the present disclosure when stretched, FIG. 15 is a schematic view of the surgical robotic arm when bent, and FIG. 16 is a partially enlarged schematic view of the surgical robotic arm at its wrist joint. As illustrated, the surgical robotic arm comprises: a flexible arm 2; an terminal manipulator 4 to perform surgical operations, i.e., to provide main functions of a surgical instrument; a wrist joint 3, two ends of which are respectively connected to the flexible arm 2 and the terminal manipulator 4. A control cable of the terminal manipulator 4 is accessed from the flexible arm 2, passes through the wrist joint 3 and is connected to the terminal manipulator 4. Among them, the flexible arm 2 may be the above-described flexible arm.

The terminal manipulator 4 may include a jaw, a laser scalpel, an endoscope, and the like. In order to improve the flexibility of the terminal manipulator 4, the wrist joint 3 may involve the freedoms of movement in multiple directions. Since the wrist joint 3 is located at an end of the surgical robotic arm, it will not be easily affected by the driving tendons 6 of other joints. With excellent control performance of the terminal manipulator 4 and the flexible arm 2, the surgical robotic arm disclosed in the present disclosure can accurately and efficiently perform various operations in the single-port access surgery.

In the embodiment illustrated in FIG. 16, the wrist joint 3 comprises: a terminal connecting section 33 for connection with the terminal manipulator 4; a flexible section 31 including a second turn 311 provided in multiple segments, two ends of the flexible section 31 being respectively connected to the flexible arm 2 and the terminal connecting section 33; and a central backbone 32 passing through the center of the flexible section 31, two ends of which are respectively connected to the terminal connecting section 33 and the flexible arm 2.

Similar to the supporting section 11, the terminal connecting section 33 may have a certain rigidity to play a supporting role. The shape, structure and material of the second turn 311 may be the same as or different from the first turn 121. That is to say, the second turn 311 may involve a concentric ring or disc structure as shown in FIG. 3, or a helical structure formed by connecting a plurality of turns with each other in a helical manner, or a structure shown in FIGS. 5 to 10 with a contact aided part 122. At each second turn 311 of the flexible section 31, at least two pairs of tendon through-holes 5 are oppositely provided for the driving tendon 6 to pass through. The flexible section 31 is endowed with a freedom of movement in two directions by the at least two pairs of tendon through-holes 5, so that the terminal manipulator 4 can move and operate flexibly.

Optionally, the central backbone 32 passing through the center of the flexible section 31 may have elasticity. Since the central backbone 32 is provided in the flexible section 31 near the distal end, the driving force of the driving tendon 6 is not required to be high. Where the elastic central backbone 32 is incorporated and the freedom of movement of the flexible section 31 is ensured, the elastic restoring force of the central backbone 32 can be used to prevent the misalignment of the flexible section 31, and the reliability of the surgical robotic arm can be further improved.

In an embodiment, the flexible arm 2 and the wrist joint 3 (at least partially, the end connecting section 33 thereof) in the surgical robotic arm can be integrally formed by 3D printing. In actual use, it is only necessary to install movable element(s) of the separate terminal manipulator 4 and the driving tendon 6 onto main part(s) of the surgical robotic arm produced via 3D printing, and then the assembly process is completed. Therefore, the use of 3D printing can improve manufacturing efficiency, extend service life and reduce costs. In 3D printing, the same or different materials may be used to form various parts of the surgical robot arm, such as the flexible joint 1, the decoupling section 2, the flexible section 31, the central backbone 32, and the terminal connecting section 33.

Finally, while the present disclosure has been described in detail in connection with only a limited number of embodiments, it should be readily understood that the present disclosure is not limited to such disclosed embodiments. Rather, the present disclosure can be modified to incorporate any number of variations, alterations, substitutions or equivalent arrangements not heretofore described, but which are commensurate with the spirit and scope of the present disclosure. Additionally, while various embodiments of the present disclosure have been described, it is to be understood that aspects of the present disclosure may include only some of the described embodiments. Accordingly, the present disclosure is not to be seen as limited by the foregoing description, but is only limited by the scope of the appended claims.

1. A flexible joint (1) comprising two support sections (11) and an articulated section (12) connected between the two support sections (11), the articulated section (12) including a plurality of first turns (121) with contact aided parts (122);
wherein the contact aided parts (122) are oppositely arranged on two sides of each first turn (121), and when the flexible joint is in a bent state, the contact aided parts (122) of adjacent first turns (121) are in contact with each other; and
wherein at each first turn (121) of the articulated section (12) are provided a plurality of tendon through-holes (5) for a driving tendon (6) to pass through.

2. The flexible joint (1) according to claim 1, wherein the plurality of first turns (121) are connected with each other in a helical manner to form a helical structure.

3. The flexible joint (1) according to claim 1, wherein the contact aided parts (122) of adjacent first turns (121) are in rolling contact with each other or mesh with each other.

4. The flexible joint (1) according to claim 3, wherein each of the contact aided parts (122) is formed as a smooth protrusion toward an adjacent contact aided part (122), and is in tangential contact with the adjacent contact aided part (122) at the tip of the protrusion.

5. The flexible joint (1) according to claim 4, wherein the contact aided part (122) has a circular or elliptical cross-section.

6. The flexible joint (1) according to claim 5, wherein the contact aided part (122) is configured as a cylinder or a cone.

7. The flexible joint (1) according to claim 3, wherein each of the contact aided parts (122) is formed as a tooth-like structure meshing with an adjacent contact aided part (122), or has a polygonal cross-section.

8. The flexible joint (1) according to claim 1, wherein an axial centerline of the contact aided part (122) coincides with a circumferential centerline of the first turn (121).

9. The flexible joint (1) according to claim 1, wherein a connection line between at least one pair of tendon through-holes (5) among the plurality of tendon through-holes (5) is perpendicular to an axial centerline of the contact aided part (122).

10. The flexible joint (1) according to claim 1, wherein the flexible joint (1) is integrally formed by 3D printing.

11. The flexible joint (1) according to claim 1, wherein the tendon through-holes through-hole (5) are each is designed to be open at the periphery of the first turn (121).

12. A flexible arm (2), comprising:
at least two flexible joints (1) according to claim 1; and
a decoupling section (21), which is disposed between two adjacent flexible joints (1) and connected to the respective support sections (11) of the two adjacent flexible joints (1).

13. The flexible arm (2) according to claim 12, wherein a tendon route (22) is provided on the decoupling section (21), and extends to a tendon through-hole (5) provided at a support section (11) of the flexible joint (1) for a driving tendon (6) to pass through.

14. The flexible arm (2) according to claim 13, wherein the tendon route (22) is helically provided on a surface of the decoupling section (21).

15. The flexible arm (2) according to claim 13, wherein the tendon route (22) includes a helical wire groove provided on an outer surface of the decoupling section (21).

16. The flexible arm (2) according to claim 12, wherein the decoupling section (21) is configured as a cylinder.

17. The flexible arm (2) according to claim 13, wherein the corresponding tendon through-holes (5) of two adjacent flexible joints (1) are offset from each other.

18. The flexible arm (2) according to claim 17, wherein the tendon through-holes (5) in the support sections (11) of the two adjacent flexible joints (1) are positioned in such a way that the two adjacent flexible joints (1) bend in an S form in one plane.

19. The flexible arm (2) according to claim 13, wherein when an outer surface of the decoupling section (21) is deployed in a plane, the tendon route (22) forms an S-shaped curve in the plane.

20. The flexible arm (2) according to claim 12, wherein the flexible arm (2) is integrally formed by 3D printing.

21. A surgical robotic arm, comprising:
a flexible arm (2) according to claim 12;
a terminal manipulator (4) to perform surgical operations; and
a wrist joint (3), two ends of which are respectively connected to the flexible arm (2) and the terminal manipulator (4),
wherein a control cable of the terminal manipulator (4) is accessed from the flexible arm (2), passes through the wrist joint (3) and connects with the terminal manipulator (4).

22. The surgical robotic arm according to claim 21, wherein the wrist joint (3) comprises:
a terminal connecting section (33) for connection with the terminal manipulator (4);
a flexible section (31) including a second turn (311) provided in multiple segments, two ends of which are respectively connected to the terminal connecting section (33) and the flexible arm (2), wherein at each second turn (311) of the flexible section (31) are oppositely provided at least two pairs of tendon through-holes (5) for a driving tendon (6) to pass through; and
a central backbone (32) with elasticity passing through the center of the flexible section (31), two ends of which are respectively connected to the terminal connecting section (33) and the flexible arm (2).

23. The surgical robotic arm according to claim 21, wherein the flexible arm (2) and the wrist joint (3) are integrally formed by 3D printing.
